(19) United States
(12) Patent Application Publication
(10) Pub. No.: US 2021/0205163 A1
(43) Pub. Date: Jul. 8, 2021

(54) DEVICE AND METHOD FOR POSITIONING A JOINT

(71) Applicant: Trice Medical, Inc., Malvern, PA (US)

(72) Inventors: Sean McMillan, Mullica Hill, NJ (US); Alfred J. Intintoli, West Chester, PA (US); Benjamin R. Brozanski, Philadelphia, PA (US)

(21) Appl. No.: 17/273,127

(22) PCT Filed: Sep. 23, 2019
(86) PCT No.: PCT/US2019/052441

§ 371 Date: Mar. 3, 2021

Related U.S. Application Data
(60) Provisional application No. 62/736,382, filed on Sep. 25, 2018.

(51) Int. Cl. A61G 13/12; A61G 13/00; A61B 1/00; A61B 1/317; A61B 5/00

(52) U.S. Cl. CPC A61G 13/1285; A61G 13/0063; A61G 13/0072; A61G 2200/322; A61B 1/317; A61B 5/702; A61B 1/00078

(57) ABSTRACT

The present invention relates to a joint positioning device for positioning a joint and methods for using such devices. The joint positioning device may comprise a patient support comprising a substantially flat surface, the patient support configured to be positioned on a medical examination table; a first leg and a second leg extending from the patient support, the legs configured to immobilize the patient support to the medical examination table in a lateral or longitudinal direction. The joint positioning device may further comprise a vertical pillar or pole, the vertical pillar or pole oriented substantially perpendicular to the substantially flat surface of the patient support, the vertical pillar or vertical pole extending in an opposite direction from the first leg and the second leg.

### CROSS-REFERENCE TO RELATED APPLICATIONS

This application claims priority to U.S. Provisional Application No. 62/736,382, entitled DEVICE AND METHOD FOR POSITIONING A JOINT, filed on Sep. 25, 2018, the disclosure of which is hereby incorporated by reference in its entirety.

### BACKGROUND OF THE INVENTION

### Field of the Invention

This application describes examples of apparatuses, methods, and systems for the positioning of a patient for proper visualization and access of tissues for injection or treatment.

### Description of the Related Art

Traditional therapeutic and diagnostic surgical procedures for pathologies located within the body can cause significant trauma to the intervening tissues and potentially the target tissue site. Such procedures often require a long incision, extensive muscle stripping, prolonged retraction of tissues, denervation and devascularization of tissue. Such procedures may require operating room time of several hours followed by several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for orthopedic applications due to the need for access to locations within the body and the danger of damage to vital intervening tissues. While developments in minimally invasive surgery are steps in the right direction, there remains a need for further development in minimally invasive surgical instruments and methods.

Treatment of internal tissue sites, such as the treatment of an orthopedic joint, often requires proper positioning for visualization of and access to the target internal tissues for imaging and injections. Currently in the field, it is often difficult for a surgeon to access specific areas of the anatomy and thus they often fail to visualize and diagnose completely. However, when a patient is properly positioned, a surgeon or doctor may be able to access specific areas of the anatomy for proper diagnosis. With proper positioning, typically tight or inaccessible tissue sites within the patient can be opened up for visualization and access. Additionally, orthopedic joints must be properly positioned and stabilized for access to the desired internal tissue site, for proper visualization of the desired internal tissue site and to prevent injury to the patient. Consequently, there is need for improved devices and methods for positioning of a patient to open up areas for visualization and access to an internal tissue site.

### SUMMARY

examples of the present invention relate to joint positioning devices, methods, and systems. In some examples, joint positioning devices comprise a patient support, one or more legs extending from the patient support, and a vertical pillar extending from the patient support. In some examples, the patient support may be dimensioned to fit a standard medical examination table, lengthwise or widthwise. In some examples, the one or more legs may be positioned along the length of the patient support to immobilize the patient support relative to the medical examination table.

In some examples, a joint positioning device for positioning a joint comprises:

a patient support comprising a substantially flat surface, the patient support configured to be positioned on a medical examination table;
a first leg and a second leg extending from the patient support, the legs configured to immobilize the patient support relative to the medical examination table in a lateral or longitudinal direction; and

a vertical pillar mounted to the patient support, the vertical pillar oriented substantially perpendicular to the substantially flat surface of the patient support, the vertical pillar extending in an opposite direction from the first leg and the second leg.

In some examples, the patient support is configured such that the patient may rest on the patient support. The vertical pillar may be configured to be positioned on a lateral side of a leg of the patient. The vertical pillar may be further configured to brace the patient's leg laterally. The vertical pillar may be further configured to position the patient's knee to provide access to a medial compartment of the knee for an endoscopic procedure. In some examples, at least a portion of the patient support is configured to extend from the surface of the medical examination table. In some examples, the distance between the first leg and second leg can be adjusted. The distance between the first leg and second leg may be substantially similar to the length of the medical examination table. The distance between the first leg and second leg may be substantially similar to the width of the medical examination table. In some examples, the length of the patient support is substantially aligned with the length of the medical examination table. The length of the patient support may be substantially aligned with the width of the medical examination table. In some examples, the position of the vertical pillar may be adjusted along a length of the patient support. In some examples, the vertical pillar comprises a metal pole. In some examples, the metal pole is padded.

In particular examples, a method of positioning a knee for an endoscopic procedure comprises:

providing a positioning device comprising a patient support, a first leg, a second leg, and a vertical pillar;

positioning at least a portion of a flat surface of the patient support on a medical examination table;

adjusting the second leg to immobilize the positioning device relative to the medical examination table in a lateral or longitudinal direction;

positioning a patient to rest on the flat surface of the patient support such that the vertical pillar is positioned on a lateral side of a leg of the patient;

positioning the vertical pillar on a lateral side of the leg of the patient; and

applying lateral pressure to the leg such that a medial compartment of the knee becomes open.

In some examples, the method may further comprise inserting a needle endoscope into a tissue site and collecting an image. In some examples, the method may further comprise inserting a needle endoscope into a tissue site and performing an injection. In some examples, the method may further comprise aligning a length of the patient support with a length of the medical examination table. In some examples, the method may further comprise aligning a length of the patient support with a width of the medical examination table.

In some examples, a joint positioning device for positioning a joint comprises:

a patient support comprising a substantially flat surface, the patient support configured to be positioned on a medical examination table;

a first leg and a second leg extending from the patient support, the legs configured to immobilize the patient support relative to the medical examination table in a lateral or longitudinal direction; and

a vertical pole extending from the patient support, the vertical pole oriented substantially perpendicular to the substantially flat surface of the patient support, the vertical pole extending in an opposite direction from the first leg and the second leg.

In some examples, the patient support is configured such that the patient may rest on the patient support. The vertical pillar may be configured to be positioned behind the patient. The vertical pillar may further comprise a strap configured to support the patient's wrist and extend the patient's shoulder. The vertical pole may be further configured to position the patient's shoulder to provide access to a glenoid cavity of the shoulder for an endoscopic procedure. In some examples, at least a portion of the patient support is configured to extend from the surface of the medical examination table. In some examples, the distance between the first leg and second leg can be adjusted. The distance between the first leg and second leg may be substantially similar to the length of the medical examination table. The distance between the first leg and second leg may be substantially similar to the width of the medical examination table. In some examples, the length of the patient support is substantially aligned with the length of the medical examination table. The length of the patient support may be substantially aligned with the width of the medical examination table. In some examples, the position of the vertical pole may be adjusted along a length of the patient support. In some examples, the vertical pole may be telescoping.

In particular examples, a method of positioning a shoulder for an endoscopic procedure comprises:

providing a positioning device comprising a patient support, a first leg, a second leg, and a vertical pole;

positioning at least a portion of a flat surface of the patient support on a medical examination table;

adjusting the second leg to immobilize the positioning device relative to the medical examination table in a lateral or longitudinal direction;

positioning a patient to rest on the flat surface of the patient support such that the vertical pole is positioned behind the patient and such that the patient extends their shoulder to reach the vertical pole; and

applying pressure to the shoulder such that the glenoid cavity of the shoulder becomes open.

In some examples, the method may further comprise inserting a needle endoscope into a tissue site and collecting an image. In some examples, the method may further comprise inserting a needle endoscope into a tissue site and performing an injection. In some examples, the method may further comprise aligning a length of the patient support with a length of the medical examination table. In some examples, the method may further comprise aligning a length of the patient support with a width of the medical examination table.

Additional examples of joint positioning devices and their methods of use, are described below.

### BRIEF DESCRIPTION OF THE DRAWINGS

Other features and advantages of the present invention will be apparent from the following detailed description of the invention, taken in conjunction with the accompanying drawings of which:

FIGS. 1A-1C illustrate an example of a joint positioning device positioned on a medical examination table from different perspectives.

FIGS. 2A-2D illustrate an example of a joint positioning device. FIGS. 2B-2D illustrate various views of an example of a joint positioning device.

FIG. 3 illustrates a close-up view of the vertical pillar of a joint positioning device.

FIG. 4 illustrates a close-up view of an example of the vertical pillar of a joint positioning device with the outer padding removed.

FIG. 5A illustrates an example of a joint positioning device positioned on a medical examination table with a patient seat.

FIG. 5B illustrates an example of a patient seat fastening to a joint positioning device.

FIG. 6 illustrates a patient positioned on a joint positioning device.

FIGS. 7A-7C illustrate another example of a joint positioning device positioned on a medical examination table. FIGS. 7B-7C illustrate an embodiment of a joint positioning device positioned on a medical examination table from different perspectives.

FIGS. 8A-8D illustrates an example of the joint positioning device.

FIGS. 8B-8D illustrate various views of an example of the joint positioning device. FIG. 8D illustrates a bottom view of an example of the joint positioning device showing a stabilizer plate.

FIG. 9 illustrates another example of joint positioning device positioned on a medical examination table with a patient pad.

FIG. 10 illustrates another example of the joint positioning device.

FIG. 11 illustrates a patient positioned on a joint positioning device.

### DETAILED DESCRIPTION

Examples disclosed in this section or elsewhere in this application relate to devices and methods for positioning joints for minimally invasive tissue visualization and access, including endoscopic procedures.

Before the present invention is described in greater detail, it is to be understood that this invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the terms “about,” “around,” and “approximately.” These terms are used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual examples described and illustrated herein has discrete components and features which may be readily separated from or combined with the features of any of the other several examples without departing from the scope or spirit of the present invention. Any recited method can be carried out in the order of events recited or in any other order which is logically possible.

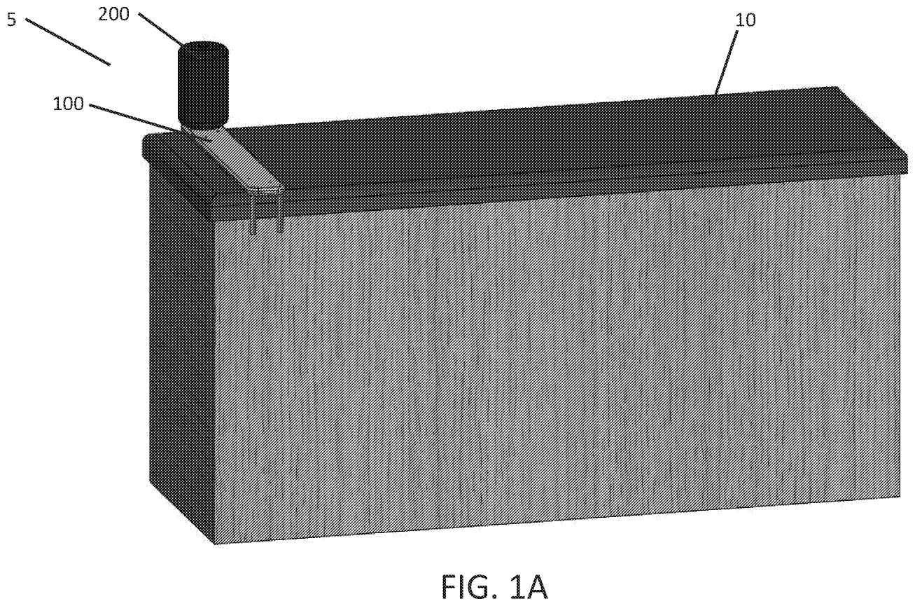

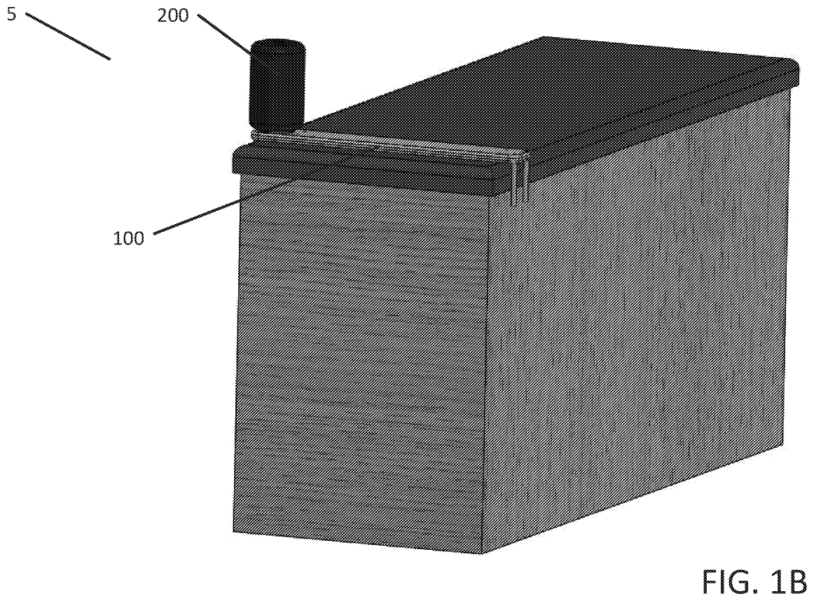

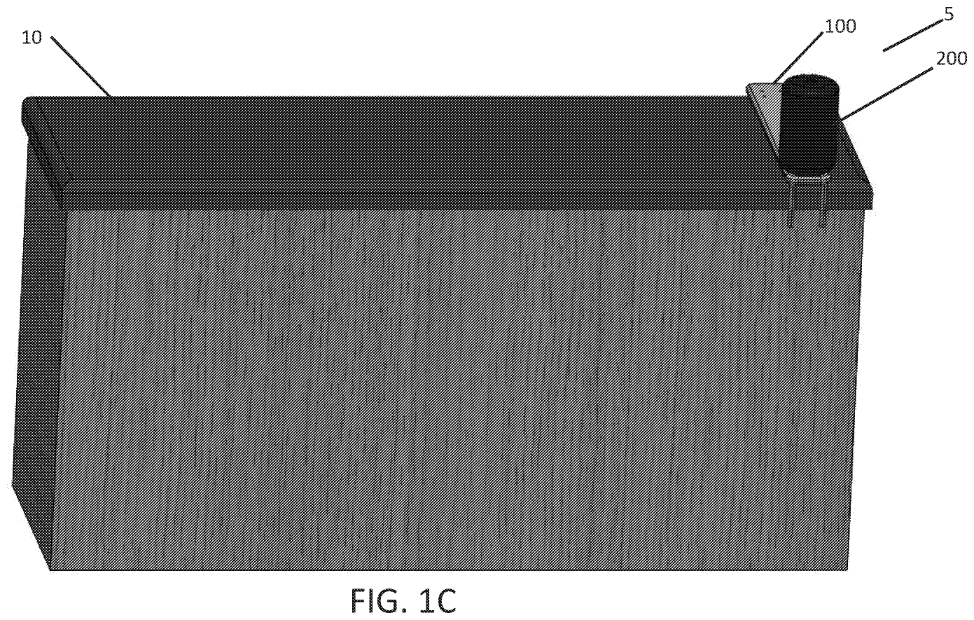

FIG. 1A-1C illustrate an example of a joint positioning device 5 positioned on a medical examination table 10. FIGS. 1B-1C illustrate an example of a joint positioning device 5 positioned on a medical examination table 10 from different perspectives. According to an example, the joint positioning device 5 can be adapted for proper positioning and stabilization of a valgus knee for imaging and injection. However, in examples, the joint positioning device 5 is applicable to numerous other medical treatments of various joints. The joint positioning device 5 may also be used to access different areas or tissue sites of any particular joint, for example the knee and the shoulder. In some examples, the device may be suitable for the wrist, ankle, hip, elbow, or other appropriate joints.

In some examples, the joint positioning device 5 includes a patient support 100 and a vertical pillar 200. In some examples, the joint positioning device 5 may be positioned on a medical examination table 10 widthwise, as shown in FIGS. 1A-1C. In other examples, the joint positioning device 5 may be positioned on a medical examination table 10 lengthwise. In particular examples, the joint positioning device 5 may be positioned on a standard medical examination table 10. In some examples, the joint positioning device 5 may be positioned on other various types and sizes of medical examination tables 10, such as surgical or operating tables, bariatric tables, pediatric exam tables, or treatment tables.

In certain examples, the patient support 100 may be a substantially flat board or platform. The patient support 100 may be substantially rigid. In other examples, the patient support 100 may be flexible. In some examples, the patient support 100 may be made of plastic, metal, foam, for example closed-cell foams, or other suitable material. In certain examples, the metal may be stainless steel, chrome, or any suitable metal that is corrosion-resistant and readily cleanable.

The patient support 100 may be positioned to lay flat on the surface of a medical examination table 10. In some examples, the patient support 100 may be substantially rectangular, as shown in FIGS. 1A-1C. In some examples, the patient support 100 may be elongated, extending along a longitudinal axis.

In examples, the patient support 100 may be dimensioned to fit an examination medical table 10. In some examples, the patient support 100 may be adjustable such that the length or width may be adjusted.

While the dimensions of the patient support 100 may vary, in certain examples the length of the patient support 100 ranges from about 10 inches to about 100 inches, such as about 59.5 inches to about 75.5 inches, including about 20 inches to about 60 inches. For example, 26 inches or 33 inches. While the dimensions of the patient support 100 may vary, in certain examples the width of the patient support 100 ranges from 1 inch to 30 inches, including 2 inches to 20 inches, and including 3 inches to 5 inches. While the dimensions of the patient support 100 may vary, in certain examples the thickness or height of the patient support 100 ranges from 0.1 inches to 5 inches, such as 0.3 inches to 3 inches, including 0.5 inches to 1.5 inches.

The patient support 100 may have a longitudinal axis and lateral axis, where the longitudinal axis and lateral axis are substantially perpendicular from each other. The longitudinal axis may be substantially parallel to the length of the patient support 100. The lateral axis may be substantially parallel to the width of the patient support 100.

The medical examination table 10 may have a longitudinal axis and lateral axis, where the longitudinal axis and lateral axis are substantially perpendicular from each other. The longitudinal axis may be substantially parallel to the length of the medical examination table 10. The lateral axis may be substantially parallel to the width of the medical examination table 10.

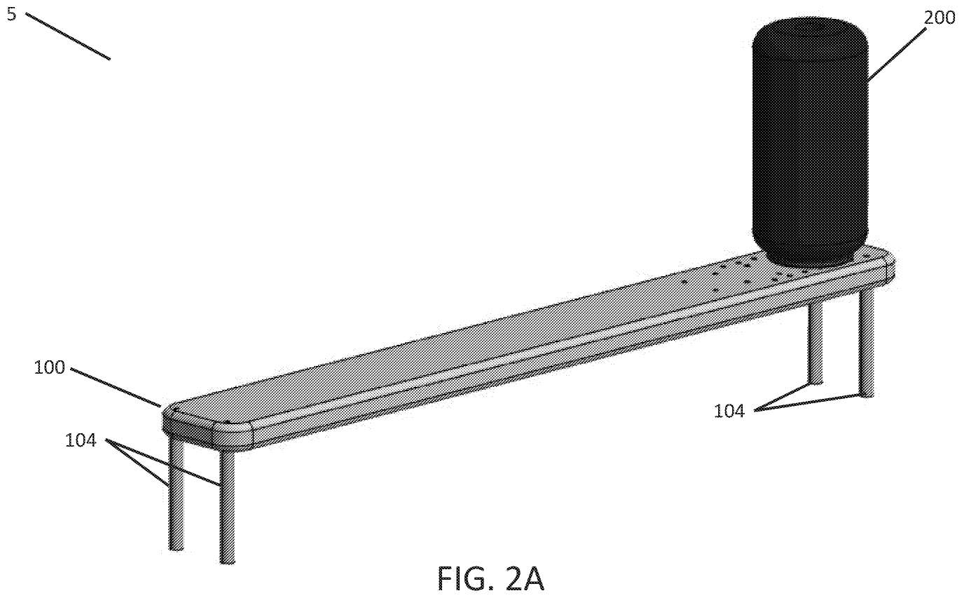

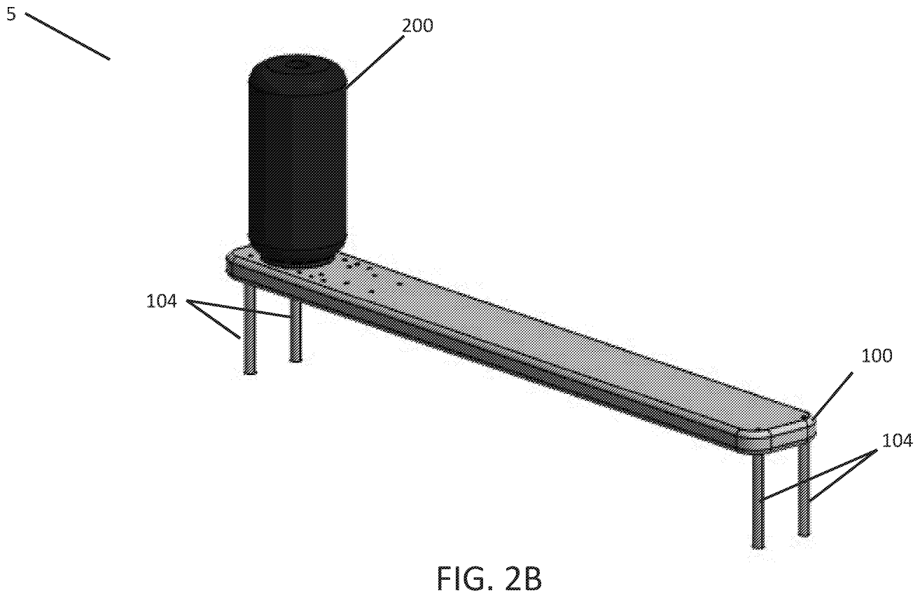

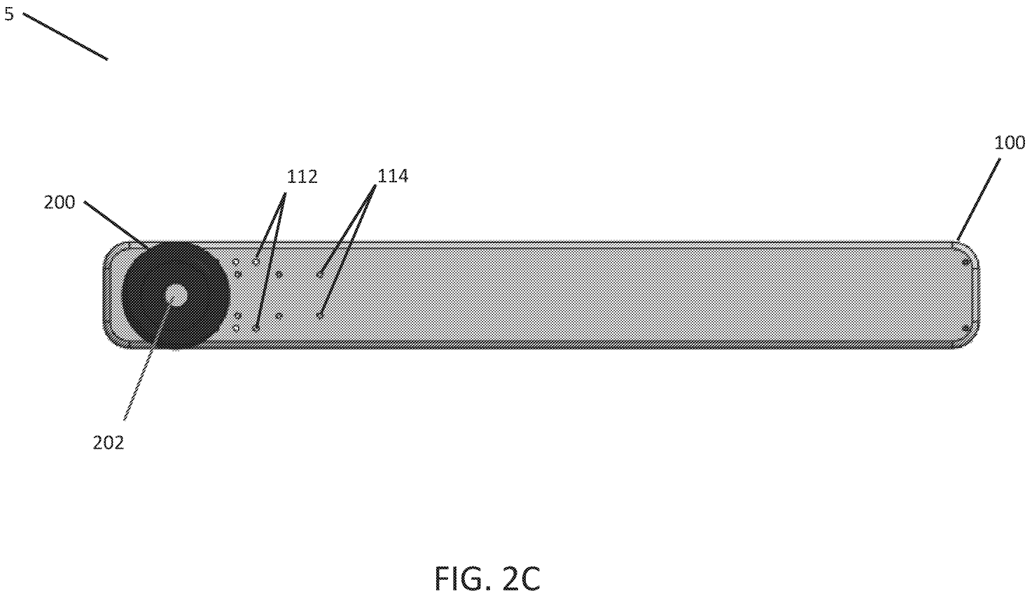

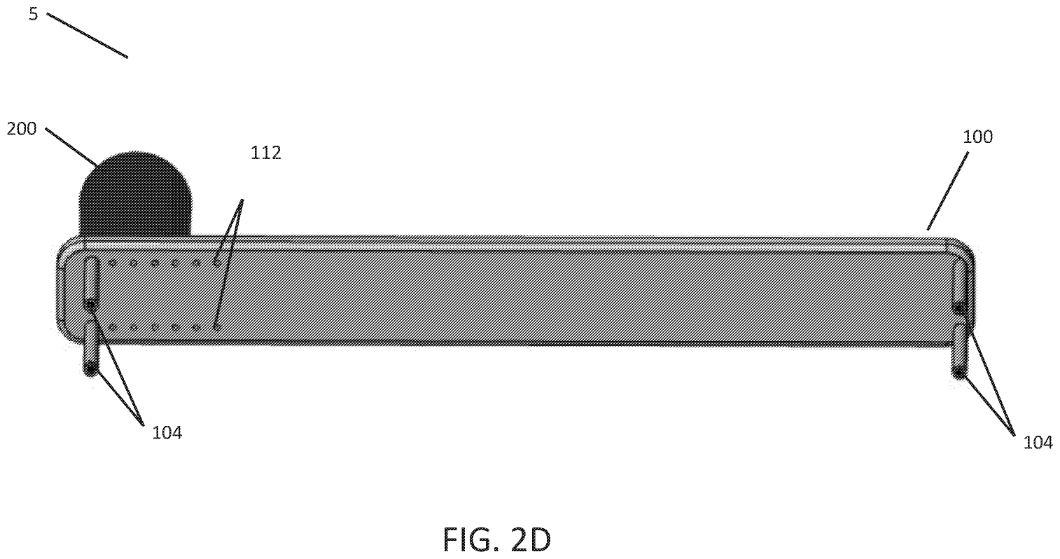

Turning to FIGS. 2A-2D, in some examples, the joint positioning device 5 includes one or more legs 104 that extend from the substantially flat surface of the patient support 100. In some examples, the one or more legs 104 may be substantially perpendicular from the surface of the patient support 100. In some examples, the joint positioning device 5 includes four legs 104, as shown in FIGS. 2A-2D. In certain examples, the joint positioning device 5 includes no legs 104, one leg 104, two legs 104, three legs 104, four legs 104, six legs, eight legs, ten legs, twelve legs, or more than twelve legs. The one or more legs 104 may be positioned at the ends or corners of the patient support 100, as shown in FIGS. 2A-2D.

In some examples, the patient support 100 may have a series of leg holes 112, as shown in FIGS. 2C-2D. The leg holes 112 may receive the one or more legs 104. The leg holes 112 may be threaded or otherwise structured to engage and receive the one or more legs 104. In some examples, the legs may be press-fit into the leg hole. In certain examples, the leg may be attached via a bayonet attachment. The one or more legs 104 may have corresponding threads or structure to engage with the threads or structure of the leg holes 112 of the patient support 100. As shown in FIGS. 2C-2D, the leg holes 112 may be positioned at the proximal and/or distal end of the patient support 100.

Furthermore, the patient support 100 may include a series of leg holes 112 along the length of the patient support 100. The series of leg holes 112 allows for the legs 104 to be adjusted and positioned along the length of the patient support 100, such that the distance between the legs 104 may be adjusted. In some examples, the distance between the legs 104 to correspond to the width or length of the medical examination table 10. In some examples, the distance between the legs 104 may be adjusted to correspond to the length of width of a standard medical examination table 10 or a variety of different sized medical examination tables 10.

The legs 104 may immobilize the patient support 100 relative to the medical examination table 10 in a lateral or longitudinal direction. The legs 104 may laterally immobilize the joint positioning device 5 to the medical examination table 10.

For example, in some examples, two legs 104 are positioned at the distal end of the patient support 100. The patient support 100 may then be placed widthwise or lengthwise on a medical examination table 10. Two other legs 104 may be adjusted and positioned at the proximal end of the patient support 100, corresponding to the width or length of the medical examination table 10. This will immobilize the patient support 100 such that it cannot be shifted in a lateral direction, relative to the lateral or longitudinal axis of the medical examination table 10. The legs 104 may immobilize the patient support 100 relative to the medical examination table 10 in a lateral or longitudinal direction. The legs 104 may laterally immobilize the joint positioning device 5 to the medical examination table 10.

For example, if a pediatric medical examination table 10 is used, which has a smaller width and length than a standard medical examination table 10, the one or more legs 104 may be adjusted such that they are positioned in a different set of leg holes 112. The legs 104 may be positioned and distanced relative to each other correspond to the width or length of the pediatric medical examination table 10 to immobilize the patient support 100.

In some examples, the joint positioning device 5 may be used on a medical examination table 10 in different configurations. For example, the legs 104 of the joint positioning device in one configuration may be positioned to fit a medical examination table 10 lengthwise. The same joint positioning device 5 in another configuration may then be used to fit the medical examination table 10 widthwise, by adjusting the legs 104, without adjusting the length or dimensions of the patient support 100. For example, the legs 104 of the same joint positioning device 5 may be spaced from each other the correspond to the width of the medical examination table 10.

In some examples, a joint positioning device 5 may be used on a variety of medical examination tables 10. For example, the legs 104 of the joint positioning device 5 in one configuration may be positioned to fit a standard medical examination table 10, lengthwise or widthwise. The same joint positioning device 5 in another configuration may then be used to fit a pediatric table, widthwise or lengthwise, by adjusting the legs 104, without adjusting the length or dimensions of the patient support 100. For example, the legs 104 of the same joint positioning device 5 may be spaced from each other the correspond to the length or width of the pediatric medical examination table 10.

In some examples, the joint positioning device 5 may not include legs 104. The joint positioning device 5 may be placed on the medical examination table 10. In some examples, the joint positioning device 5 may be attached or secured to the medical examination table 10 by other means.

In some examples, one leg 104 may be attached to the patient support 100. In some examples, two legs 104 may be attached to the patient support 100, one leg 104 on the proximal end and distal end of the patient support 100.

In some examples, three legs 104 may be attached to the patient support 100, with one leg 104 on the proximal end and distal end of the patient support 100. The third leg 104 may be on either lateral end of the patient support 100. The third leg 104 may also be positioned anywhere on the patient support 100 to further stabilize or immobilize the joint positioning device 5. In some examples, the first leg 104 may be positioned on a first end of the patient support 100, the second leg 104 may be positioned on a second end of the patient support 100, and the third leg 104 may be positioned on a third end of the patient support 100. The joint positioning device 5 may be immobilized along a first axis on both sides and partially immobilized along a second axis, where the second axis is substantially perpendicular to the first axis.

In some examples, the joint positioning device 5 may include four legs 104, as shown in FIGS. 2A-2B. In some examples, four legs 104 may be attached to the patient support 100, two legs 104 on the proximal end and two legs 104 on the distal end of the patient support 100. In some examples, the joint positioning device 5 may include more than four legs 104.

The one or more legs 104 do not have to be attached to the patient support 100 before placing the patient support 100 on the medical examination table 10. For example, two legs 104 may first be positioned at a first end of the patient support 100, such as the distal end of the patient support 100. The patient support 100 may then be placed in a desired orientation, such as widthwise or lengthwise, on the medical examination table 10. Then, two other legs 104 may be adjusted and positioned at a second end of the patient support 100, such as the proximal end of the patient support 100. The second set of legs 104 may be attached to the patient support 100 at a distance from the first set of legs 104, where the distance or space between the legs 104 corresponds to the length or width of the medical examination table 10.

The legs 104 may be secured to the two ends of the patient support 100, such that they are positioned at the lateral ends or proximal and distal ends of the medical examination table 10. The legs 104 will immobilize the patient support 100, such that the joint positioning device 5 cannot be shifted in a longitudinal direction relative to the medical examination table 10.

The legs 104 may be secured to the two ends of the patient support 100, such that they are positioned at the longitudinal ends of the medical examination table 10. The legs 104 will immobilize the patient support 100, such that the joint positioning device 5 cannot be shifted in a lateral direction relative to the medical examination table 10.

In other examples, all four legs 104 may first be attached to the patient support 100, two legs 104 on the proximal and distal ends of the patient support 100. The patient positioning device 5 may then be placed on the medical examination table 10, with the two legs 104 on the lateral ends or the proximal and distal ends of the medical examination table 10.

Although the series of leg holes 112 shown in FIGS. 2C-2D are positioned on the proximal end of the patient support 100, the series of leg holes 112 may be positioned anywhere along the length of the patient support 100. In some examples, the series of leg holes 112 may be positioned on the proximal and distal ends or lateral ends of the patient support 100. In some examples, the series of leg holes 112 may be positioned along the entire length of the patient support 100.

As described above, the patient support 100 may be positioned on the medical examination table 10 widthwise. The length of the patient support 100 may be substantially aligned with the width of the medical examination table 10. The longitudinal axis of the patient support 100 may be aligned with the lateral axis of the medical examination table 10. The patient support 100 may have a length substantially similar to the width of the medical examination table 10. In some examples, the one or more legs 104 may be positioned on the longitudinal sides or ends of the medical examination table 10, such that the one or more legs 104 immobilize the patient support 100, as shown in FIGS. 1A-1C. In some examples, the one or more legs 104 may be positioned on the longitudinal sides or ends of the medical examination table 10, such that the patient support 100 is immobilized relative to the longitudinal axis of the patient support 100, as shown in FIGS. 1A-1C. In some examples, the one or more legs 104 may be positioned on the longitudinal sides or ends of the medical examination table 10, such that the patient support 100 is immobilized along the lateral axis of the medical examination table 10, as shown in FIGS. 1A-1C.

In some examples, the patient support 100 may be positioned on the lateral sides of the standard examination table 10. In some examples, the patient support 100 may be positioned on the proximal end of the examination table 10, as shown in FIGS. 1A-1B. In other examples, the patient support 100 may be positioned on the distal end of the examination table 10 or anywhere on the examination table 10.

As described above, the patient support 100 may be positioned on the medical examination table 10 lengthwise. The length of the patient support 100 may be substantially aligned with the length of the medical examination table 10. The longitudinal axis of the patient support 100 may be aligned with the longitudinal axis of the medical examination table 10. The patient support 100 may have a length substantially similar to the length of the medical examination table 10. In some examples, the one or more legs 104 may be positioned on the distal and proximal ends or lateral ends of the medical examination table 10, such that the patient support 100 is immobilized relative to the lateral axis of the medical examination table 10. In some examples, the one or more legs 104 may be positioned on the lateral sides or ends of the medical examination table 10, such that the patient support 100 is immobilized along the longitudinal axis of the medical examination table 10.

In some examples, the patient support 100 may be positioned on the medical examination table 10 lengthwise. The longitudinal axis of the patient support 100 may be aligned with the longitudinal axis of the medical examination table 10. In some examples, the one or more legs 104 may be positioned on the distal and proximal ends of the medical examination table 10, such that the one or more legs 104 immobilize the patient support 100 relative to the longitudinal axis of the medical examination table 10. The patient support 100 may have a length substantially similar to the length of the medical examination table 10. In some examples, the patient support 100 may be positioned on the lateral sides of the standard examination table 10.

In some examples, the patient support 100 may be positioned at the edge of the medical examination table 10. In some examples, the patient support 100 may be positioned towards the middle of the medical examination table 10. In some examples, the patient support 100 may be positioned away from the edge of the medical examination table 10.

In some examples, the patient support 100 may be entirely positioned on the surface of the medical examination table 10. In some examples, the patient support 100 may rest partially on the surface of the medical examination table 10 and partially extending from the surface of the medical examination table 10.

While the dimensions of the one or more legs 104 may vary, in certain examples the length of the one or more legs 104 ranges from 2 inches to 32 inches, such as 3 inches to 16 inches, including 4 inches to 8 inches. In some examples, the one or more legs 104 may be adjustable such that the length of the one or more legs 104 may be adjusted shorter or longer.

In some examples, the one or more legs 104 may be substantially straight, as shown in FIGS. 2A-2D. In other examples, the one or more legs 104 may include protrusions such as hooks or horizontal rods that extend at the distal end of the one or more legs 104. In some examples, protrusions may be integral with the one or more legs 104. In some examples, the protrusions may be attached to the one or more legs 104. In some examples, the one or more legs 104 may have protrusions that extend perpendicularly from the length of the one or more legs 104. In some examples, the one or more legs 104 may be connected to the patient support 100 at the proximal end of the one or more legs 104 and the protrusions are at the distal end of the one or more legs 104.

The protrusions may include hooks or horizontal rods. The protrusions may secure the patient support 100 to the medical exam table 10 by engaging or fastening to the medical examination table 10. Some medical examination tables 10 may have a padded seat or surface, storage drawers or cabinets, and other features. The one or more legs 104 may have protrusions to engage with the features of the medical examination table 10. For example, the one or more legs 104 may have hooks that engage with the side and underside of the padded surface or seat of the medical examination table 10, such that the patient support 100 is vertically immobilized.

Once the joint positioning device 5 is positioned and placed on the medical examination table 10, the patient may sit partially on the medical examination table 10 and partially on the patient support 100, such that the patient's legs are placed over the patient support 100. In some examples, the patient support 100 may be dimensioned such that the patient may rest entirely on the patient support 100.

In some examples, the patient support 100 may have a minimal height or thickness to allow the patient to rest their legs comfortably over the patient support 100. In some examples, the patient support 100 may have a substantial height or thickness to support the weight of the patient. In some examples, the patient support 100 may have a height or thickness that is optimized to support or position the patient's legs. In some examples, the patient support 100 may have a height or thickness that is optimized to position the patient's leg for access and visualization for an endoscopic procedure. In some examples, the patient support 100 itself maybe padded or lined for the comfort of the patient.

As discussed above, the joint positioning device 5 may be entirely positioned on the surface of the medical examination table 10. The patient may sit with their legs positioned off the end or edge of the medical examination table 10. The patient may sit with their legs resting against the medical examination table 10.

As discussed above, the joint positioning device 5 may be positioned partially on the surface of the medical examination table 10 and partially extending off the medical examination table 10. The patient may sit with their legs positioned over the patient support 100 with their legs positioned off the edge of the patient support 100.

### Joint Positioning

In some examples, the joint positioning device 5 includes a vertical pillar 200 that extends substantially perpendicular from the substantially flat surface of the patient support 100. In certain examples, the vertical pillar 200 extends substantially perpendicular from the substantially flat surface of the patient support 100, in a direction opposite from the one or more legs 104.

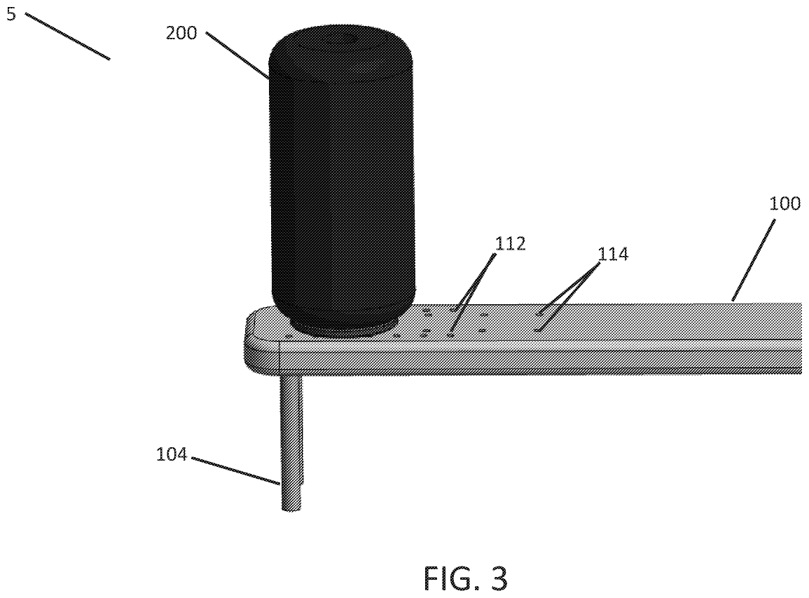

The vertical pillar 200 may be positioned at the end of the patient support 100, as shown in FIGS. 2A-2D. In examples, the vertical pillar 200 may be positioned at either lateral end of the patient support 100, such as the proximal or distal end of the patient support 100. In some examples, the vertical pillar 200 may be positioned anywhere along the length and/or width of the patient support 100. In some examples, the vertical pillar 200 may be padded. The vertical pillar 200 may include a pole 204 with padding or a padded surface, as shown in FIG. 3.

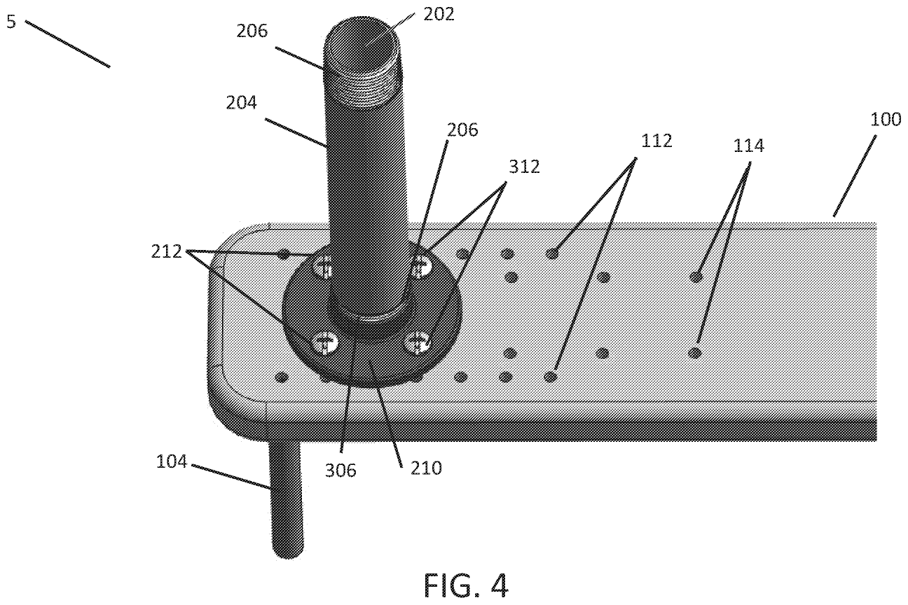

FIG. 4 illustrates the vertical pillar 200 without the padding or a padded surface. In some examples, the vertical pillar 200 may be a pole 204 without padding. In examples, the pole 204 may be made of metal, plastic, or any other suitable material. The padding or padded surface may be made of foam, felt, or any other suitable material.

While the dimensions of the pole 204, in certain examples the height of the pole 204 ranges from 4 inches to 30 inches, such as 5 inches to 15 inches, including 6 inches to 10 inches. While the dimensions of the pole 204 may vary, in certain examples the diameter of the pole 204 ranges from 0.3 inches to 6 inches, such as 0.5 inches to 3 inches, including 0.7 inch to 1.5 inches.

The vertical pillar 200 may include a base 210. The base 210 may include a central hole or aperture 306 to receive the pole 204. While the dimensions of the base 210 may vary, in certain examples the diameter of the base 210 ranges from 1 inches to 15 inches, such as 2 inches to 10 inches, including 3 inches to 5 inches.

The pole 204 may include a central hole or aperture 202. The ends 206 of the pole 204 may be threaded or otherwise structured to engage and secure the pole 204 to the base 210. The ends 206 of the pole 204 may be threaded or otherwise structured to engage center hole or aperture 306 of the base 210. The center hole or aperture 306 of the base 210 may have corresponding threading or structure to engage with the threads of the ends 206 of the pole 204. One of skill in the art will understand that the pole may be secured into base via any suitable means disclosed herein this section or elsewhere in the specification. For example, the pole may be secured via press-fit and/or via bayonet attachment.

The pillar holes 114 of the patient support 100 may be threaded or otherwise structured to secure the pole 204 to the patient support 100. The screws or pegs 312 may be inserted through the base 210 and into the patient support 100. The base 210 may include a series of holes or apertures 212 configured to receive screws or pegs 312. The screws or pegs 312 may be used to secure the vertical pillar 200 through the base 210 to the patient support 100.

In some examples, the patient support 100 may have a series of pillar holes 114, as shown in FIGS. 2C-2D. As shown in FIG. 4, the pillar holes 112 may receive screws or pegs 312 to secure the vertical pillar 200 to the patient support 100. The pillar holes 114 may be threaded or otherwise structured to engage the screws or pegs 312 and secure the vertical pillar 200 to the patient support 100. As shown in FIGS. 2C-2D, the pillar holes 114 may be positioned on the proximal end of the patient support 100. The series of pillar holes 114 allows for the vertical pillar 200 to be positioned along of the length of the patient support 100.

In particular examples, the vertical pillar 200 may be integral with the patient support 100. The vertical pillar 200 may be attached or secured to the patient support 100 in a number of ways. The vertical pillar 200 may attach directly to the patient support 100 without the base 210. For example, the patient support 100 may have a hole dimensioned to receive the vertical pillar 200. The vertical pillar 200 may have threads or be structured to engage with a hole of the patient support 100.

The vertical pillar 200 may be positioned on the patient support 100 such that the vertical pillar 200 may be placed on a lateral side of the patient's leg of interest.

While the dimensions of the vertical pillar 200 may vary, in certain examples the height of the vertical pillar 200 ranges from 4 inches to 30 inches, such as 5 inches to 15 inches, including 6 inches to 10 inches. While the dimensions of the vertical pillar 200 may vary, in certain examples the diameter of the vertical pillar 200 ranges from 2 inches to 12 inches, such as 4 inches to 10 inches, including 5 inches to 6 inches.

In some examples, the vertical pillar 200 may be round as shown in FIGS. 3 and 4. In some examples, the vertical pillar 200 may have variety of shapes, such as contoured round pincushion (vertical lines go toward center), contoured round barrel (vertical lines go away from center), rectangular or square, or any other suitable shape.

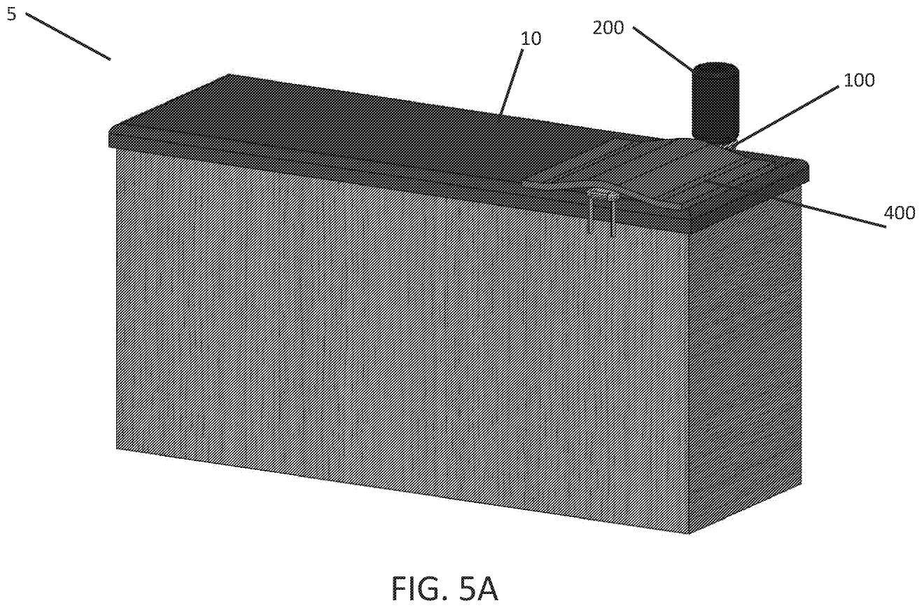

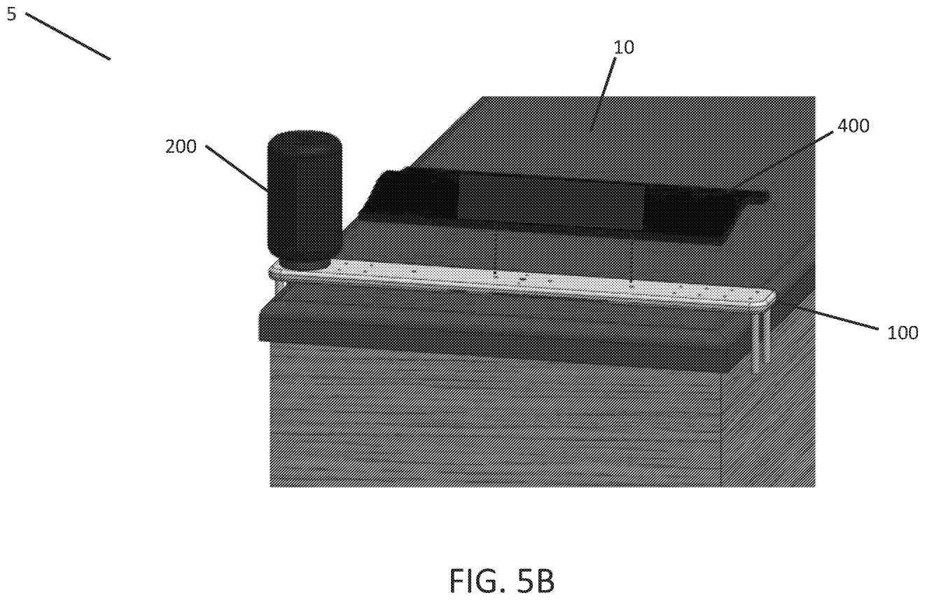

FIG. 5A illustrates an example of a joint positioning device 5 positioned on a medical examination table 10 with a patient seat 400. As shown in FIG. 5A, the joint positioning device 5 may include a patient seat 400 for the patient. The patient may rest on the patient seat 400 and/or the patient support 100. The patient seat 400 may be provided for the comfort of the patient. The patient may be seated on the patient seat 400. The patient seat 400 may be integral with the patient support 100. The patient seat 400 may be attached or connected to the patient support 100. The patient seat 400 may be adjustable based on the joint positioning device 5 on the medical examination table 10. The patient seat 400 may also be adjustable based on the position of the vertical pillar 200 on the patient support 100. For example, the patient seat 400 may be placed anywhere along the length of the patient support 100, such that the patient seat 400 can be adjusted depending on the position of the patient and the vertical pillar 200. The patient seat 400 may assist the physician in appropriately positioning the patient in relation to the vertical pillar 200 and the patient support 100.

FIG. 5B illustrates an example of a patient seat 400 fastening to a joint positioning device. As shown in FIG. 5B, the patient seat 400 may be secured to the patient support 100. The patient seat 400 can include one or more fasteners (not shown) to be received within one or more holes of the patient support 100. The fasteners can be connected at least to the bottom surface of the patient seat 400. The fasteners can be aligned with and be received within the one or more holes of the top surface of the patient support 100.

While the dimensions of the patient seat 400 may vary, in certain examples the width of the patient seat 400 ranges from 5 inches to 34 inches, such as 6 inches to 24 inches, including 7 inches to 9 inches. While the dimensions of the patient seat 400 may vary, in certain examples the length of the patient seat 400 or the patient support 100 ranges from about 10 inches to about 100 inches, such as about 59.5 inches to about 75.5 inches, including about 20 inches to about 60 inches. While the dimensions of the patient seat 400 may vary, in certain examples the height or thickness of the patient seat 400 ranges from 0.1 inches to 5 inches, such as 0.3 inches to 3 inches, including 0.5 inches to 1.5 inches.

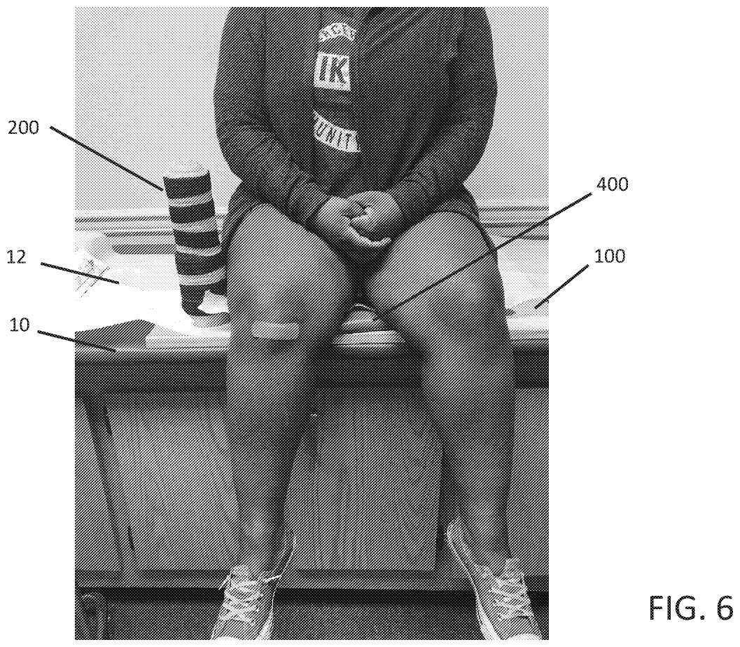

FIG. 6 illustrates an example of a patient positioned on a joint positioning device. Once the joint positioning device is positioned and placed on the medical examination table 10, the patient may sit on the medical examination table 10 and the patient support 100, such that the patient's legs are placed over the patient support 100. The patient may sit on to the joint positioning device, immediately adjacent to or next to the vertical pillar 200, as illustrated in FIG. 6.

In certain examples, the patient's legs may be positioned over the patient support 100 and extend from the side of the medical examination table 10. The patient's leg of interest may be braced against the vertical pillar 200. Once the patient's leg is braced against the vertical pillar 200, a physician may press, push, or apply pressure to the patient's leg laterally. The physician may position or place the patient's leg in an outward direction to open up the medical compartment of the patient's knee. While the patient's lower leg is being pushed laterally or outwardly, the patient's upper leg remains stabilized by the vertical pillar 200 to put a slight torque or force on the patient's knee. The patient may be positioned on either side of the vertical pillar 200 to allow for access of either leg or either knee of the patient.

In particular examples, once the medial compartment of the patient's knee is in an open position, the physician may then have the necessary access and visualization of the medial compartment of the patient's knee to examine the internal tissue of the knee, such as via a needle endoscope. The physician may insert a needle endoscope into a tissue site and collect an image. Additionally, once the medial compartment of the patient's knee is open, the physician may and perform any necessary procedures or treatments such as an injection. The physician may then inject the patient's joint, such as via a needle endoscope, with access to the medial compartment of the knee.

### Shoulder Positioning Device

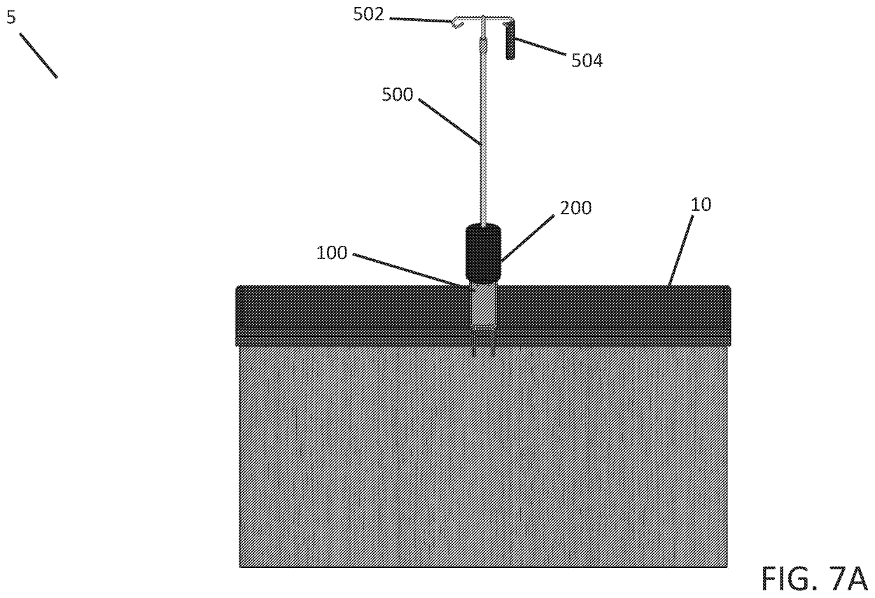

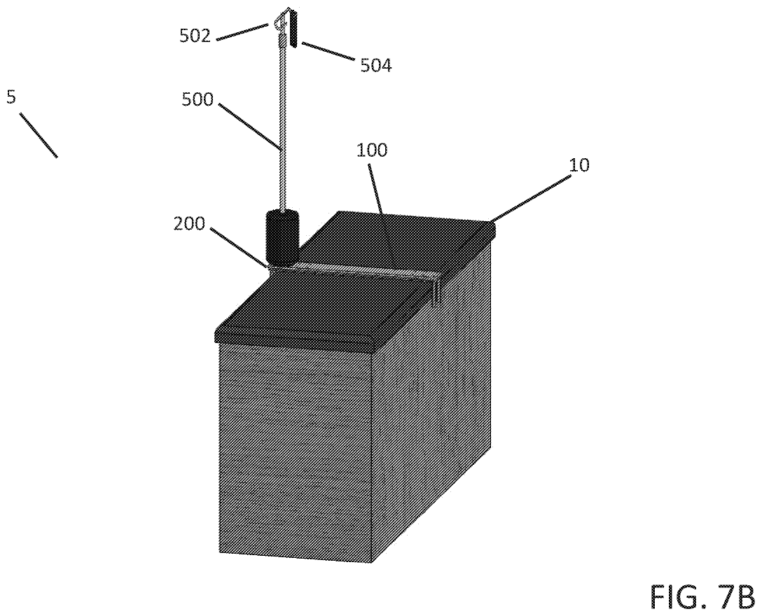

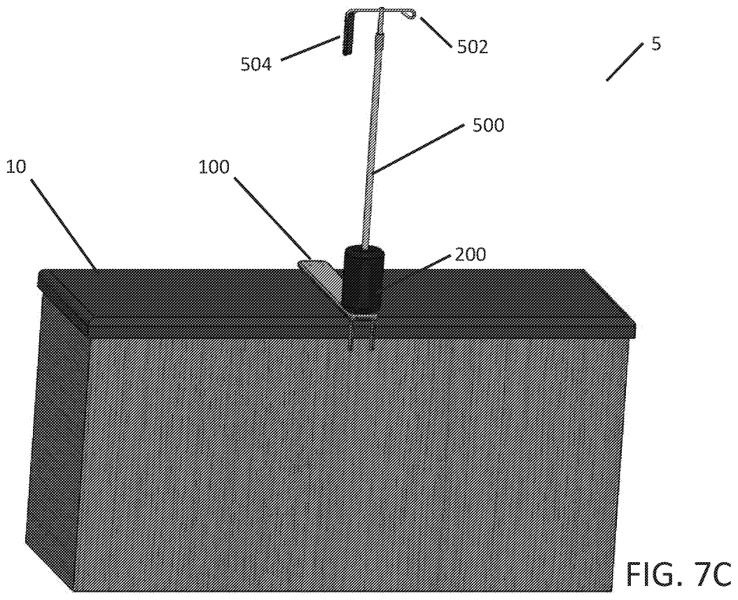

FIGS. 7A-7C illustrate another example of a joint positioning device 5 positioned on a medical examination table 10. FIGS. 7B-7C illustrate the example of a joint positioning device 5 positioned on a medical examination table 10 from different perspectives. According to an example, the joint positioning device 5 can be adapted for proper positioning and stabilization of a shoulder joint for imaging and injection.

The joint positioning device 5 illustrated in FIGS. 7A-7C may be similar to the joint positioning device 5 described above in FIGS. 1-6, including the patient support 100, the vertical pillar 200, the legs 104, and how the joint positioning device 5 can be positioned on or immobilized relative to the medical examination table 10. In some examples, the joint positioning device 5 includes a patient support 100 and a vertical pillar 200.

According to the example illustrated in FIGS. 7A-7C, the joint positioning device 5 can be adapted for proper positioning and stabilization of a shoulder joint for imaging and injection. In some examples, the joint positioning device 5 may include a patient support 100, a vertical pillar 200, a shoulder pole 500, and a strap 504.

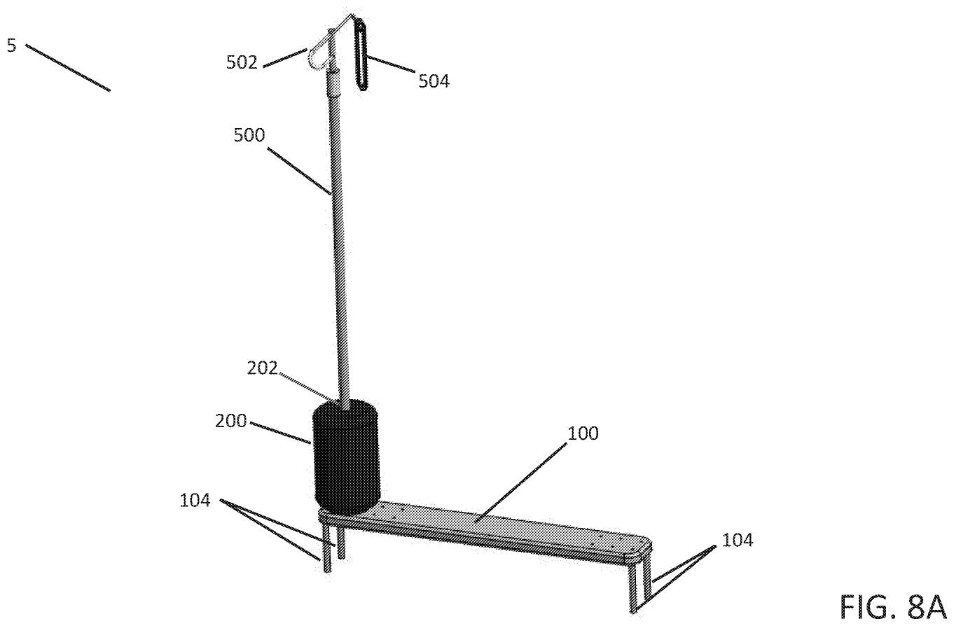

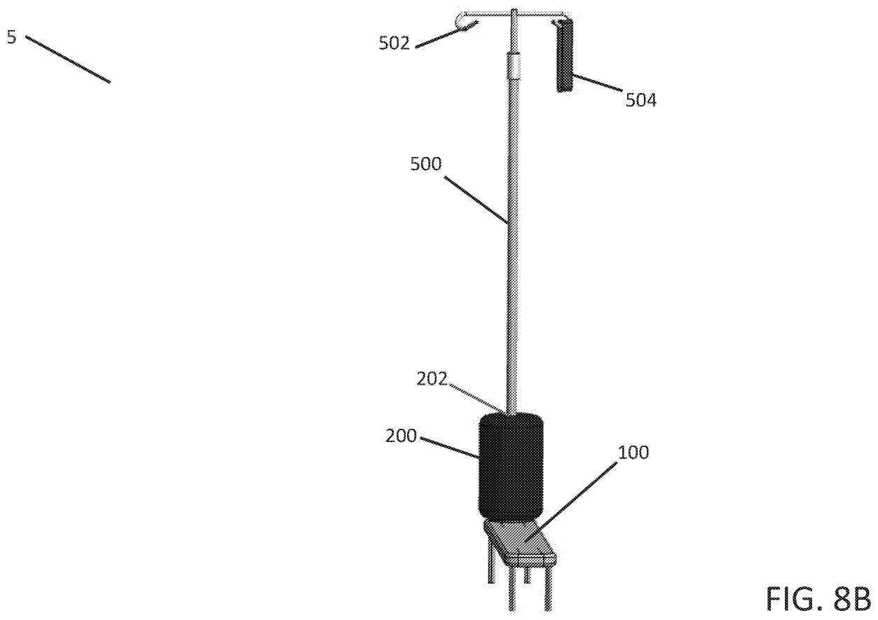

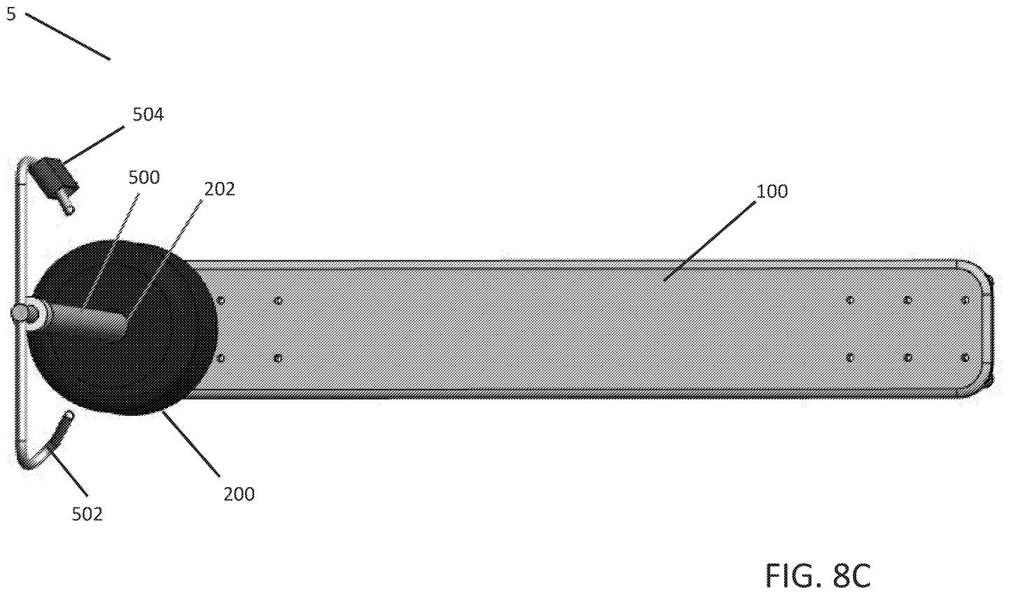

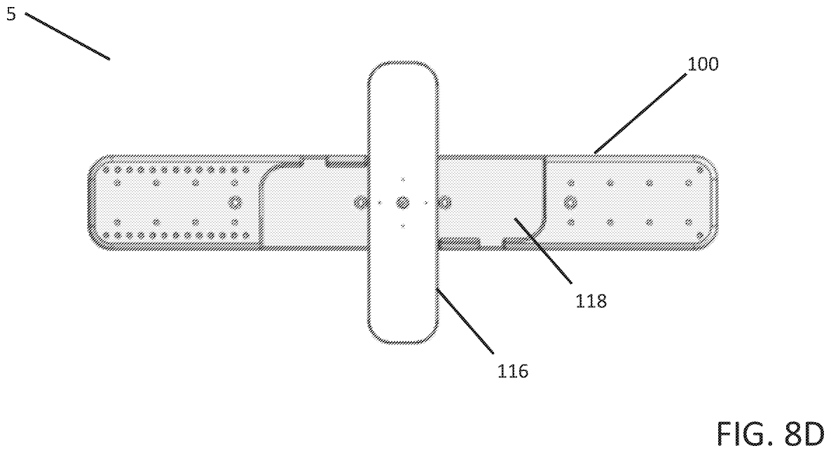

As shown in FIGS. 8A-8D, the shoulder pole 500 can be positioned in the hole or aperture 202 of the pole 204, such that the shoulder pole 500 may extend vertically from the vertical pillar 200. As described previously, the pole 204 of the vertical pillar 200 may include a central hole or aperture 202.

The shoulder pole 500 may be secured to the joint positioning device 5 in several ways. In one example, the shoulder pole 500 may be inserted into the hole or aperture 202 of the pole 204. In some examples, the hole or aperture 202 of the pole 204 may be threaded or otherwise engaged to secure the pole 500. The pole 500 may have corresponding threads or structure to engage with the threads or structure of the hole or aperture 202 of the pole 204. In some examples, the shoulder pole 500 may attach to the patient support 100. The patient support 100 may have a hole dimensioned to receive the shoulder pole 500. The shoulder pole 500 may have threads or be structured to engage with the patient support 100. The shoulder pole 500 may be attached inserted into the hole or aperture 202 of the vertical pillar 200 and attach to the patient support 100. The shoulder pole 500 may also attach directly to the patient support 100 without the vertical pillar 200 present. One of skill in the art will understand that the pole may be secured via any

The shoulder pole 500 may be integral with the patient support 100. The shoulder pole 500 may be inserted or engage with the leg holes 112 or pillar holes 114 of the patient support 100. The shoulder pole 500 may have a base, similar to the base 210 of the vertical pillar 200, that may be secured to the patient support 100.

The shoulder pole 500 can be telescoping such the height of the shoulder pole 500 can be raised or lowered. The length or height of the shoulder pole 500 can be adjusted depending on the patient's size or orientation.

While the dimensions of the shoulder pole 500 may vary, in certain examples the length or height of the shoulder pole 500 ranges from 20 inches to 80 inches, such as 25 inches to 70 inches, including 30 inches to 60 inches. While the dimensions of the shoulder pole 500 may vary, in certain examples the diameter of the shoulder pole 500 ranges from 0.2 inches to 5 inches, such as 0.3 inches to 3 inches, including 0.5 inch to 1.5 inches.

The shoulder pole 500 can also include at least one hook 502. In some examples, the shoulder pole 500 can include one or more hooks 502. As shown in FIGS. 7A-7C, the shoulder pole 500 can include two hooks 502. As shown in FIGS. 7A-7C, the longitudinal axis or length of the hook 502 may be aligned with the longitudinal axis or length of the medical examination table 10. The hook 502 may be rotated such that the longitudinal axis or length of the hook 502 may be aligned along different axes, for example, aligned with the width or lateral axis of the medical examination table 10. The hook 502 may be positioned or rotated to correctly position the patient's arm and shoulder.

While the dimensions of the hook 502 may vary, in certain examples the length of the hook 502 ranges from 4 inches to 30 inches, such as 6 inches to 20 inches, including 9 inches to 11 inches. While the dimensions of the hook 502 may vary, in certain examples the height of the hook 502 ranges from 0.5 inches to 5 inches, such as 1.0 inches to 4 inches, including 1.5 inches to 2.5 inches.

The joint positioning device 5 can also include a strap 504. The strap 504 can be attached to the one or more hooks 502. The strap 504 may be adjustable such that it can be moved to different hooks 502. The strap 504 may be adjustable such that it can be moved to along the length of the one or more hooks 502. The strap 504 may be adjustable such that the length can be adjusted depending on the patient's size or orientation.

While the dimensions of the strap 504 may vary, in certain examples the length of the strap 504 ranges from 2 inches to 30 inches, such as 4 inches to 20 inches, including 6 inches to 10 inches.

FIG. 8D illustrates a bottom view of an example of the joint positioning device 5 showing a stabilizer plate 116. A stabilizer plate 116 may be placed within a recess 118 between the proximal and distal ends of the patient support 100. The longitudinal axis or length of the stabilizer plate 116 may be aligned with the longitudinal axis or length of the patient support 100. The stabilizer plate 116 can rotate to be perpendicular to a longitudinal axis or length of the patient support 100. The stabilizer plate 116 may be used in various positions to stabilize and counterbalance the pillar 200 and/or the shoulder pole 500. As shown in FIG. 8D, the joint positioning device 5 may be used without the legs 104. Although the stabilizer plate 116 is shown in context of the joint positioning device 5 for shoulder positioning, this stabilizer plate 116 can also be used in all examples of the joint positioning device 5, such as the joint positioning device 5 for positioning a patient's knee in FIGS. 1A-6.

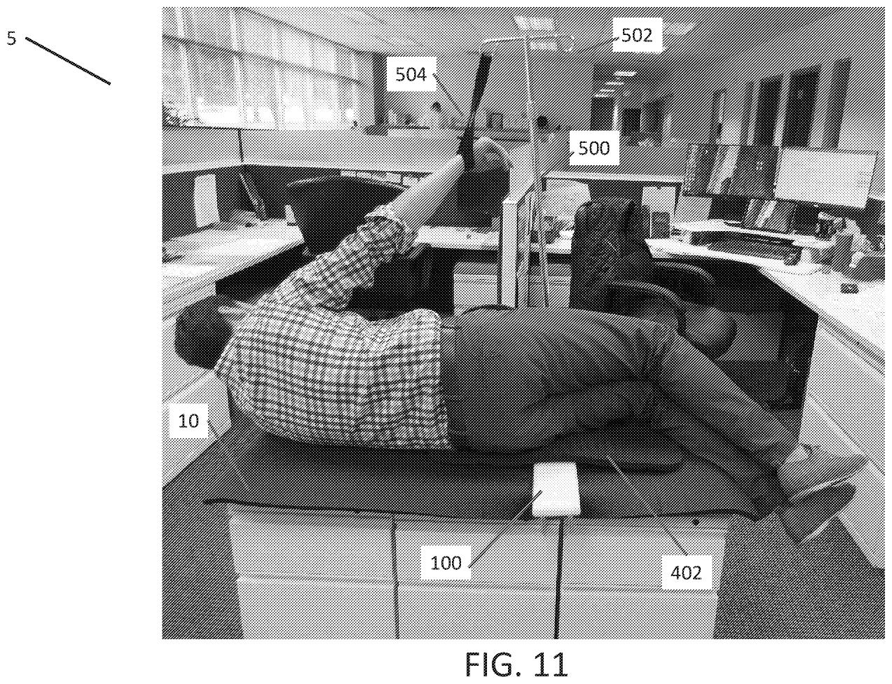

FIG. 11 illustrates an example of a patient positioned on a joint positioning device 5. Once the joint positioning device 5 is positioned and placed on the medical examination table 10, the patient may lay on their side on the medical examination table 10 and/or the patient support 100. The patient may be in a lateral side position. The patient may lay down on the side opposite of the shoulder of interest. The patient may be positioned to lay on either of their sides to allow for access of either shoulder of the patient. For example, as shown in FIG. 11, the patient may lie on their left side, such that the patient's right shoulder can be examined. The patient may be positioned with the patient's hips positioned on the patient support 100. In some examples, the patient support 100 may be positioned anywhere under the patient from the patient's upper thigh to the patient's lower waist. The patient may be positioned in relation to the joint positioning device 5 and the medical examination table 10 in various ways. In some examples, the patient may lay on their back, lay on their back, sit upright, or in other various positions.

The strap 502 may hang or be attached to the hook 502, which extends from or is attached to the shoulder pole 500. As shown in FIG. 11, the shoulder pole 500 may be positioned in front of the patient. The patient may reach forward with the arm and shoulder of interest. The patient may reach forward towards the strap 504. In some examples, the patient may grab the strap 504 with their hand. In certain examples, the patient may position and rest their hand through the strap 504 such that the patient's wrist is supported by the strap 504, as shown in FIG. 11. The height of the shoulder pole 500 may be adjusted such that the patient can reach back to extend their arm and rotate their shoulder to reach the strap 504.

In particular examples, the shoulder pole 500 may be positioned behind the patient. The patient may reach behind them with the arm and shoulder of interest. The patient may reach behind them towards the strap 504. In some examples, the patient may grab the strap 504 with their hand. In other examples, the patient may position and rest their hand through the strap 504 such that the patient's wrist is supported by the strap 504. The height of the shoulder pole 500 may be adjusted such that the patient can reach back to extend their arm and rotate their shoulder to reach the strap 504.

The physician may appropriately adjust the patient, strap 504, hook 502, shoulder pole 500, and/or the patient support 100 such that the patient's arm is appropriately extended and the patient's shoulder is appropriately rotated in the correct position. The physician may position or place the patient's shoulder in an outward direction to open up the glenoid cavity of the patient's shoulder. The physician may apply pressure to the shoulder such that the glenoid cavity of the shoulder becomes open. Once the patient is in the correct position, the glenoid cavity of the shoulder of interest is accessible. While the patient's shoulder and arm is being rotated or extended, the patient's upper body remains stabilized to put a slight torque or force on the patient's shoulder.

Once the glenoid cavity of the patient's shoulder is opened, the physician may then have the necessary access and visualization of the glenoid cavity of the patient's shoulder to examine the internal tissue of the shoulder, such as via a needle endoscope. The physician may insert a needle endoscope into a tissue site and collect an image. Additionally, once the glenoid cavity of the patient's shoulder is accessible, the physician may perform any necessary procedures or treatments such as an injection. The physician may then inject the patient's joint, such as via a needle endoscope, with access to the glenoid cavity of the patient's joint.

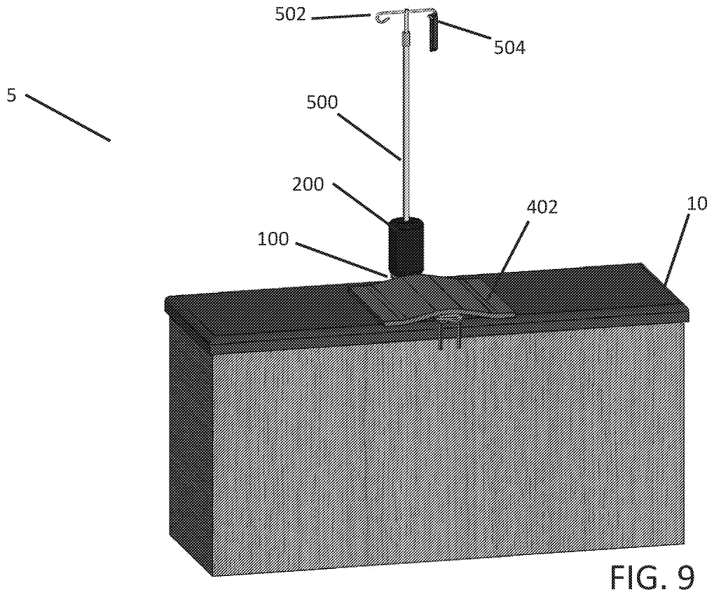

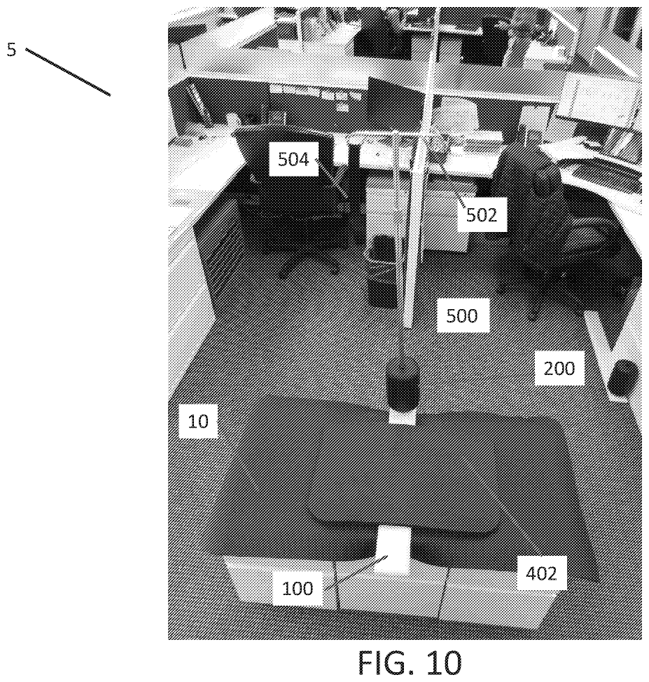

As shown in FIGS. 9-10, in examples, the joint positioning device 5 may include a patient pad 402, similar to the patient seat 400 illustrated in FIG. 5. The patient may rest on the patient pad 402 and/or the patient support 100. The patient pad 402 may be provided for the comfort of the patient.

In certain examples, the patient pad 402 may be integral with the patient support 100. The patient pad 402 may be attached or connected to the patient support 100. The patient pad 402 may be adjustable based on the joint positioning device 5 on the medical examination table 10. The patient pad 402 may also be adjustable based on the position of the vertical pillar 200 and/or shoulder pole 500 on the patient support 100. For example, the patient pad 402 may be placed anywhere along the length of the patient support 100, such that the patient pad 402 can be adjusted depending on the position of the patient, the vertical pillar 200, and/or the shoulder pole 500. The patient pad 402 may be provided for the comfort of the patient. The patient pad 402 may assist the physician in appropriately positioning the patient in relation to the shoulder pole 500, the hooks 502, the strap 504, and/or the patient support 100.

While the dimensions of the patient pad 402 may vary, in certain examples the width of the patient pad 402 ranges from 5 inches to 34 inches, such as 6 inches to 24 inches, including 7 inches to 9 inches. While the dimensions of the patient pad 402 may vary, in certain examples the length of the patient pad 402 or the patient support 100 ranges from about 10 inches to about 100 inches, such as about 59.5 inches to about 75.5 inches, including about 20 inches to 60 inches. While the dimensions of the patient pad 402 may vary, in certain examples the height or thickness of the patient pad 402 ranges from 0.1 inches to 5 inches, such as 0.3 inches to 3 inches, including 0.5 inches to 1.5 inches.

### Patient Interface

Also shown in FIG. 6, in examples, the patient joint device 5 may also be covered by a patient interface 12, such as a paper drape. The patient interface 12 may be for repeated use, one-time use and/or be disposable. The patient interface 12 may be designed to lay over and cover the patient support 100 and/or the patient seat 400, as well as lay over and cover the examination table 10. Once the environment is draped with the patient interface 12, the patient may then sit on top of the patient interface 12. The patient interface 12 may be used to maintain the sterility and minimize the contamination of the patient as well as the environment, such as the medical examination table 10 and the patient joint device 5.

In some examples, the patient interface 12 may be include a dispenser to hold a roll of paper drape. In some examples, the dispenser may be attached to the end of the medical examination table 10. A nurse or physician may unroll a portion of the paper drape from the dispenser to patient interface 12 to be draped over the patient support 100, the seat 400 (if present), and the medical examination table 10.

In some examples, the patient interface 12 may include a cut out or hole, which may allow the vertical pillar 200 to pass through. In some examples, the cut out may be rectangular, square, or some other suitable shape. The cut out may be dimensioned to receive the vertical pillar 200. In some examples, the cut out or hole may be a circular cut out that has a diameter at least as large as the outer diameter of the vertical pillar 200.

In some examples, the patient interface 12 may have slits or cuts that allow the patient interface to be positioned over the vertical pillar 200 and be draped over the patient support 100, the patient seat 400 or patient pad 402 (if present), and the medical examination table 10. In some examples, the slits or cuts of the patient interface 12 may be arranged to extend radially from a center point. In some examples, the vertical pillar 200 and/or shoulder pole 500 may be positioned at the center of the cuts or slits of the patient interface 12. The vertical pillar 200 and/or shoulder pole 500 may be pushed through the slits or cuts of the patient interface 12, such that the patient interface 12 may lay flat, covering the base 210 of the vertical pillar 200. In some examples, the slits or cuts may have a length at least as long as the diameter of the vertical pillar 200 and/or shoulder pole 500.

In some examples, the patient interface 12 may be provided as a roll with the cut outs or portions with slits or cuts periodically spaced from each other along the patient interface 12. In some examples, the space or distance between the cut outs or portions with slits or cuts may be at least the length of the medical examination table 10.

In other examples, the patient interface 12 may be a roll of paper or other suitable material that may be attached to the vertical pillar 200. The length of the roll of paper may be aligned with the length of the vertical pillar 200. In some examples, the patient interface 12 may include a dispenser to hold a roll of paper drape, such that the nurse or physician may unroll a portion of the paper drape from one end of the dispenser and around the outer surface of the vertical pillar 200, to attach the end of the paper drape to the other end of the dispenser.

In some examples, the patient joint device 5 may include a patient interface 12 that may be designed to fit over the vertical pillar 200, like a sleeve. In some examples, the patient joint device 5 may include a patient interface 12 may be designed to cover the shoulder pole 500 and/or the hook 502, like a sleeve.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

While certain examples have been described, these examples have been presented by way of example only, and are not intended to limit the scope of protection. Indeed, the novel methods and systems described in this section or elsewhere in this specification may be embodied in a variety of other forms. Furthermore, various omissions, substitutions and changes in the form of the methods and systems described in this section or elsewhere in this specification may be made. Those skilled in the art will appreciate that in some examples, the actual steps taken in the processes illustrated and/or disclosed may differ from those shown in the figures. Depending on the example, certain of the steps described above may be removed, others may be added. Furthermore, the features and attributes of the specific examples disclosed above may be combined in different ways to form additional examples, all of which fall within the scope of the present disclosure.

Although the present disclosure includes certain examples, examples and applications, it will be understood by those skilled in the art that the present disclosure extends beyond the specifically disclosed examples to other alternative examples and/or uses and obvious modifications and equivalents thereof, including examples which do not provide all of the features and advantages set forth in this section or elsewhere in this specification. Accordingly, the scope of the present disclosure is not intended to be limited by the specific disclosures of preferred examples in this section or elsewhere in this specification, and may be defined by claims as presented in this section or elsewhere in this specification or as presented in the future.

1. A joint positioning device for positioning a joint, comprising:
a patient support comprising a substantially flat surface, the patient support configured to be positioned on a medical examination table;
a first leg and a second leg extending from the patient support, the legs configured to immobilize the patient support relative to the medical examination table in a lateral or longitudinal direction; and
a vertical pillar mounted to the patient support, the vertical pillar oriented substantially perpendicular to the substantially flat surface of the patient support, the vertical pillar extending in an opposite direction from the first leg and the second leg.

2. The joint positioning device of claim 1, wherein the patient support is configured such that the patient may rest on the patient support.

3. The joint positioning device of claim 1, wherein the vertical pillar is configured to be positioned on a lateral side of a leg of the patient.

4. The joint positioning device of claim 3, wherein the vertical pillar is further configured to brace the patient's leg laterally.

5. The joint positioning device of claim 3, wherein the vertical pillar is further configured to position a patient's knee to provide access to a medial compartment of the patient's knee for an endoscopic procedure.

6. (canceled)

7. The joint positioning device of claim 1, wherein the patient support comprises a plurality of holes on an underside of the patient support, the plurality of holes configured to receive the first leg.

8. The joint positioning device of claim 7, wherein the plurality of holes are positioned along a length of the patient support.

9. (canceled)

10. The joint positioning device of claim 1, wherein the patient support has a length substantially similar to a length of the medical examination table.

11. The joint positioning device of claim 1, wherein the patient support has a length substantially similar to a width of the medical examination table.

12. (canceled)

13. (canceled)

14. The joint positioning device of claim 1, wherein a distance between the first leg and second leg is substantially similar to a length of the medical examination table.

15. The joint positioning device of claim 1, wherein a distance between the first leg and second leg is substantially similar to a width of the medical examination table.

16. The joint positioning device of claim 1, wherein a position of the vertical pillar is configured to be adjusted along a length of the patient support.

17. The joint positioning device of claim 1, wherein the vertical pillar comprises a metal pole.

18. The joint positioning device of claim 17, wherein the metal pole is padded.

19. The joint positioning device of claim 1, further comprising a stabilizer plate positioned perpendicular to a longitudinal axis of the patient support to stabilize the joint positioning device.

20. A method of positioning a knee for an endoscopic procedure, the method comprising:
providing a positioning device comprising a patient support, a first leg, a second leg, and a vertical pillar;
positioning at least a portion of a flat surface of the patient support on a medical examination table;
adjusting the second leg to immobilize the positioning device relative to the medical examination table in a lateral or longitudinal direction;
positioning a patient to rest on the flat surface of the patient support such that the vertical pillar is positioned on a lateral side of a leg of the patient;
positioning the vertical pillar on a lateral side of the leg of the patient; and
applying lateral pressure to the leg such that a medial compartment of the knee opens.

21. The method of claim 20, further comprising inserting a needle endoscope into a tissue site and collecting an image.

22. The method of claim 20, further comprising inserting a needle endoscope into a tissue site and performing an injection.

23. The method of claim 20, further comprising aligning a length of the patient support with a length of the medical examination table.

24. The method of claim 20, further comprising aligning a length of the patient support with a width of the medical examination table.

25.-44. (canceled)
